(19) United States
(12) Patent Application Publication
(10) Pub. No.: US 2026/0272935 A1
(43) Pub. Date: Sep. 17, 2026

(54) Uses of JAK Inhibitors in the Management of Sepsis and Comorbidities

(71) Applicant: Emory University, Atlanta, GA (US)

(72) Inventors: Christina Gavegnano, Decatur, GA (US); Susan Pereira Ribeiro, Atlanta, GA (US); Sebastian Roa, Atlanta, GA (US)

(21) Appl. No.: 19/475,376

(22) PCT Filed: Apr. 15, 2024
(86) PCT No.: PCT/US2024/024679

§ 371 Date: Oct. 14, 2025

Related U.S. Application Data
(60) Provisional application No. 63/459,398, filed on Apr. 14, 2023.

(51) Int. Cl. A61K 31/519; A61K 31/437; A61K 31/4985; A61K 31/506; A61K 31/529; A61K 31/541; A61K 45/06

(52) U.S. Cl. CPC A61K 31/519; A61K 31/437; A61K 31/4985; A61K 31/506; A61K 31/529; A61K 31/541; A61K 45/06

(57) ABSTRACT

Disclosed are methods of treating, preventing, or reversing sepsis and comorbidities comprising administering an effective amount of a Janus kinases (JAK) inhibitor such as baricitinib to a patient in need thereof. In certain embodiments, the subject is at risk of, exhibiting symptoms of or diagnosed with sepsis-associated organ failure, sepsis associated encephalopathy, sepsis associated acute kidney injury, sepsis induced cardiac dysfunction, sepsis induced acute respiratory distress, and/or hepato-splanchnic dysfunction.

### CROSS-REFERENCE TO RELATED APPLICATIONS

This application claims the benefit of U.S. Provisional Application No. 63/459,398 filed Apr. 14, 2023. The entirety of this application is hereby incorporated by reference for all purposes.

### BACKGROUND

Septicemia, more commonly referred to as sepsis, is a life-threatening medical condition which is a systemic inflammatory response to a microbial infection. Sepsis often progresses into severe sepsis, indicated through “sepsis-associated organ failure”, where one or multiple organ systems begin to shut down, known as “septic shock.” Comorbidities stemming from sepsis include Sepsis Associated Encephalopathy (SAE) causing neurological injury and cognitive dysfunction; Sepsis Associated Acute Kidney Injury (S-AKI) causing renal injury; Sepsis Induced Cardiac Dysfunction (SICD) impairing myocardial circulation; Sepsis Induced Acute Respiratory Distress Syndrome (ARDS) causing lung injury and respiratory system dysfunctions; and hepato-splanchnic dysfunction (HSD), impairing gut and liver functions. The treatment of sepsis typically entails hospital care, administering intravenous (IV) fluids, use of a ventilator, and sometimes surgery to remove areas of infection. Therapeutic interventions are often limited to antibiotics and corticosteroids therapies. If a patient survives, they may require an organ transplant. Thus, there is a need to identify improved therapies.

Janus kinases (JAKs) are cytoplasmic tyrosine kinases which include JAK1, JAK2, JAK3, and TYK2. The JAKs signal through a biological pathway which regulates the transcription of several genes involved in inflammation and cancer. Tzeng et al. report targeting the JAK-STAT pathway in autoimmune diseases and cancers. Biochem Pharm, 193, 114760 (2021).

Frenck et al. report the ability of recombinant murine granulocyte-macrophage colony-stimulating factor to protect neonatal rats from septic death due to Staphylococcus aureus. J Infect Dis, 1990,162(1):109-14.

Shawky et al. report an overview of globally approved JAK inhibitors. Pharmaceutics, 2022, 14, 1001.

Hu et al. report JAK/STAT pathway signals, diseases, immunity, and therapeutic regimens. Front Bioeng Biotechnol, 2023, 11:1110765.

See also International PCT Publ. No. WO2023/244611, WO2022/251434 and WO2013/082476.

References cited herein are not an admission of prior art.

### SUMMARY

This disclosure relates to methods of treating, preventing, or reversing sepsis or sepsis associated comorbidities. In certain embodiments, this disclosure relates to methods of treating, preventing, or reversing sepsis comprising administering an effective amount of a Janus kinases (JAK) inhibitor to a patient in need thereof. In certain embodiments, the JAK inhibitor is selected from baricitinib, abrocitinib, delgocitinib, fedratinib, filgotinib, oclacitinib, pacritinib, peficitinib, ruxolitinib, tofacitinib, and upadacitinib. In certain embodiments, the JAK inhibitor is administered in combination with another drug, anti-inflammatory agent, antibacterial, or antimicrobial agent.

In certain embodiments, this disclosure relates to methods of treating, preventing, or reversing sepsis-associated organ failure comprising administering an effective amount of a Janus kinases (JAK) inhibitor to a patient in need thereof. In certain embodiments, the JAK inhibitor is selected from baricitinib, abrocitinib, delgocitinib, fedratinib, filgotinib, oclacitinib, pacritinib, peficitinib, ruxolitinib, tofacitinib, and upadacitinib. In certain embodiments, the JAK inhibitor is administered in combination with another drug, anti-inflammatory agent, antibacterial, or antimicrobial agent.

In certain embodiments, this disclosure relates to methods of treating, preventing, or reversing sepsis associated encephalopathy comprising administering an effective amount of a Janus kinases (JAK) inhibitor to a patient in need thereof. In certain embodiments, the JAK inhibitor is selected from baricitinib, abrocitinib, delgocitinib, fedratinib, filgotinib, oclacitinib, pacritinib, peficitinib, ruxolitinib, tofacitinib, and upadacitinib. In certain embodiments, the JAK inhibitor is administered in combination with another drug, anti-inflammatory agent, antibacterial, or antimicrobial agent.

In certain embodiments, this disclosure relates to methods of treating, preventing, or reversing sepsis associated acute kidney injury comprising administering an effective amount of a Janus kinases (JAK) inhibitor to a patient in need thereof. In certain embodiments, the JAK inhibitor is selected from baricitinib, abrocitinib, delgocitinib, fedratinib, filgotinib, oclacitinib, pacritinib, peficitinib, ruxolitinib, tofacitinib, and upadacitinib. In certain embodiments, the JAK inhibitor is administered in combination with another drug, anti-inflammatory agent, antibacterial, or antimicrobial agent.

In certain embodiments, this disclosure relates to methods of treating, preventing, or reversing sepsis induced cardiac dysfunction comprising administering an effective amount of a Janus kinases (JAK) inhibitor to a patient in need thereof. In certain embodiments, the JAK inhibitor is selected from baricitinib, abrocitinib, delgocitinib, fedratinib, filgotinib, oclacitinib, pacritinib, peficitinib, ruxolitinib, tofacitinib, and upadacitinib. In certain embodiments, the JAK inhibitor is administered in combination with another drug, anti-inflammatory agent, antibacterial, or antimicrobial agent.

In certain embodiments, this disclosure relates to methods of treating, preventing, or reversing sepsis induced acute respiratory distress syndrome comprising administering an effective amount of a Janus kinases (JAK) inhibitor to a patient in need thereof. In certain embodiments, the JAK inhibitor is selected from baricitinib, abrocitinib, delgocitinib, fedratinib, filgotinib, oclacitinib, pacritinib, peficitinib, ruxolitinib, tofacitinib, and upadacitinib. In certain embodiments, the JAK inhibitor is administered in combination with another drug, anti-inflammatory agent, antibacterial, or antimicrobial agent.

In certain embodiments, this disclosure relates to methods of treating, preventing, or reversing hepato-splanchnic dysfunction comprising administering an effective amount of a Janus kinases (JAK) inhibitor to a patient in need thereof. In certain embodiments, the JAK inhibitor is selected from baricitinib, abrocitinib, delgocitinib, fedratinib, filgotinib, oclacitinib, pacritinib, peficitinib, ruxolitinib, tofacitinib, and upadacitinib. In certain embodiments, the JAK inhibitor is administered in combination with another drug, anti-inflammatory agent, anti-bacterial, or anti-microbial agent.

### BRIEF DESCRIPTION OF THE SEVERAL VIEWS OF THE DRAWINGS

FIG. 1 shows data from in vitro models of unique sepsis-like immune dysregulation in key primary human macrophages (P. Aeruginosa LPS), and the effect of baricitinib towards reversal of this unique phenotype. Baricitinib significantly reduces cellular surface markers HLA-DR and CD-163, key markers of sepsis. Right; P. Aeruginosa HLA-DR Mean Fluoresce Index, Middle; P. Aeruginosa CD-163 Mean Fluoresce Index, Left; P. Aeruginosa percent of cells positive for CD-163 Marker.

FIG. 2 shows data from in vitro models of unique sepsis-like immune dysregulation in key primary human macrophages (Marcescens LPS), and the effect of baricitinib towards reversal of this unique phenotype. Baricitinib significantly reduces cellular surface markers HLA-DR and CD-163, key markers of sepsis. Right S. Marcescens HLA-DR Mean Fluoresce Index, Left; S. Marcescens CD-163 Mean Fluoresce Index.

FIG. 3 shows data from in vitro models of unique sepsis-like immune dysregulation in key primary human macrophages (K. Pneumoniae LPS), and effect of baricitinib towards reversal of this unique phenotype. Baricitinib significantly reduces cellular surface marker HLA-DR, a key marker of sepsis. K. Pneumoniae HLA-DR Mean Fluoresce Index.

FIG. 4 shows data from in vitro models of unique sepsis-like immune dysregulation in key primary human macrophages (P. Aeruginosa LPS), and the effect of baricitinib towards reversal of this unique phenotype. Activated macrophages referring to gating inclusion of cells with increased size and granularity, indicating activation. Baricitinib significantly reduces cellular surface marker HLA-DR a key marker of sepsis. Right; P. Aeruginosa HLA-DR Mean Fluoresce Index, Left; P. Aeruginosa percent of cells positive for HLA-DR Marker.

FIG. 5 shows data from in vitro models of unique sepsis-like immune dysregulation in key primary human macrophages (P. Aeruginosa LPS), and the effect of baricitinib towards reversal of this unique phenotype. Inactivated macrophages referring to gating inclusion of cells with reduced size and granularity, indicating inactivation. Baricitinib significantly reduces cellular surface marker HLA-DR a key marker of sepsis. Right; P. Aeruginosa HLA-DR Mean Fluoresce Index, Left; P. Aeruginosa percent of cells positive for HLA-DR Marker.

FIG. 6 shows data from in vitro models of unique sepsis-like immune dysregulation in key primary human macrophages (S. Marcescens LPS), and the effect of baricitinib towards reversal of this unique phenotype. Activated macrophages referring to gating inclusion of cells with increased size and granularity, indicating activation. Baricitinib significantly reduces cellular surface marker CD-163 a key marker of sepsis. S. Marcescens CD-163 Mean Fluorescence Index.

FIG. 7 shows data from in vitro models of unique sepsis-like immune dysregulation in key primary human macrophages (K. Pneumoniae LPS), and the effect of baricitinib towards reversal of this unique phenotype. Activated macrophages referring to gating inclusion of cells with increased size and granularity, indicating activation. Baricitinib significantly reduces cellular surface markers HLA-DR and CD-163, key markers of sepsis. Right; K. Pneumoniae CD-163 Mean Fluoresce Index, Left; K. Pneumoniae percent of cells positive for HLA-DR Marker.

FIG. 8 shows data from in vitro models of unique sepsis-like immune dysregulation in key primary human macrophages (K. Pneumoniae LPS), and the effect of baricitinib towards reversal of this unique phenotype. Inactivated macrophages referring to gating inclusion of cells with reduced size and granularity, indicating inactivation. Baricitinib significantly reduces cellular surface marker HLA-DR percent positive cells, a key marker of sepsis. K. Pneumoniae percent of cells positive for HLA-DR Marker.

FIG. 9 shows data from in vitro models of unique sepsis-like immune dysregulation in key primary human macrophages (E. Coli LPS), and the effect of baricitinib towards reversal of this unique phenotype. Inactivated macrophages referring to gating inclusion of cells with reduced size and granularity, indicating inactivation. Baricitinib significantly increases the percentage of inactivated macrophages compared to the no drug control.

FIG. 10 shows data from in vitro models of unique sepsis-like immune dysregulation in key primary human macrophages (K. Pneumoniae LPS), and the effect of baricitinib towards reversal of this unique phenotype. Activated macrophages referring to gating inclusion of cells with increased size and granularity, indicating activation. Inactivated macrophages referring to gating inclusion of cells with reduced size and granularity, indicating inactivation. Right; Baricitinib significantly decreases the percentage of activated macrophages compared to the no drug control; Left; Baricitinib significantly increases the percentage of inactivated macrophages compared to the no drug control.

FIG. 11 shows data from in vitro models of unique sepsis-like immune dysregulation in key primary human macrophages (S. Marcescens LPS), and the effect of baricitinib towards reversal of this unique phenotype. Activated macrophages referring to gating inclusion of cells with increased size and granularity, indicating activation. Inactivated macrophages referring to gating inclusion of cells with reduced size and granularity, indicating inactivation. Right; Baricitinib significantly decreases the percentage of activated macrophages compared to the no drug control; Left, Baricitinib significantly increases the percentage of inactivated macrophages compared to the no drug control.

FIG. 12 shows at from in vitro models of unique sepsis-like immune dysregulation in key primary human macrophages (P. Aeruginosa LPS), and the effect of baricitinib towards reversal of this unique phenotype. Activated macrophages referring to gating inclusion of cells with increased size and granularity, indicating activation. Baricitinib significantly decreases the percentage of activated macrophages compared to the no drug control.

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

An “embodiment” of this disclosure refers to an example, but not necessarily limited to such example. As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow reference will be made to a number of terms that shall be defined to have the following meanings unless a contrary intention is apparent.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. Patent law in that they are inclusive or open-ended and do not exclude additional, unrecited elements or method steps.

“Consisting essentially of” or “consists of” or the like, when applied to methods and compositions encompassed by the present disclosure refers to compositions like those disclosed herein that exclude certain prior art elements to provide an inventive feature of a claim, but which may contain additional composition components or method steps, etc., that do not materially affect the basic and novel characteristic(s) of the compositions or methods.

The terms “JAK inhibitor” or “Janus kinase inhibitor” refers to any variety of compounds, (small molecules, biological molecules, anti-JAK antibodies, interfering RNA, shRNA, nucleic acids/vectors encoding proteins, CRISPR-Cas mediated systems, etc.) that, when in contact with a Janus kinase enzyme (JAK1, JAK2, JAK3, TYK2) specifically bind with a Janus kinase enzyme altering the JAK-STAT signaling pathway. Examples include baricitinib, tofacitinib, upadacitinib cerdulatinib, gandotinib, lestaurtinib, momelotinib, pacritinib ruxolitinib, oclacitinib, peficitinib, fedratinib, filgotinib, delgocitinib, abrocitinib, all of which are contemplated for uses disclosed herein.

As used herein, the term “small molecule” refers to any variety of covalently bound molecules with a molecular weight of less than 900 or 1000 Da. Typically, the majority of atoms include carbon, hydrogen, oxygen, nitrogen, and to a lesser extent sulfur and/or a halogen. Examples include steroids, short peptides, mono or polycyclic aromatic or non-aromatic, heterocyclic compounds.

As used herein, “subject” refers to any animal, preferably a human patient.

As used herein, the terms “treat” and “treating” are not limited to the case where the subject (e.g., patient) is cured and the disease is eradicated. Rather, embodiments, of the present disclosure also contemplate treatment that merely reduces symptoms, reverses, and/or delays disease progression.

As used herein, the terms “prevent” and “preventing” include the prevention of the recurrence, spread or onset. It is not intended that the present disclosure be limited to complete prevention. In some embodiments, the onset is delayed, or the severity of the disease is reduced.

As used herein, the term “combination with” when used to describe administration of an agent with an additional treatment means such that the agent may be administered prior to, together with, or after the additional treatment, or a combination thereof, such that multiple agents are bioavailable at some overlapping time.

The term “effective amount” refers to that amount of a compound or pharmaceutical composition described herein that is sufficient to effect the intended application including, but not limited to, disease treatment, as illustrated below. In relation to a combination therapy, an “effective amount” indicates the combination of agent results in synergistic or additive effect when compared to the agents individually. The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The specific dose will vary depending on, for example, the particular compound chosen, the dosing regimen to be followed, whether it is administered in combination with other agents, timing of administration, the tissue to which it is administered, and the physical delivery system in which it is carried.

The terms “drug,” “agent,” “pharmaceutical agent,” and similar terms are used interchangeably herein, and mean and include an agent, drug, compound, composition of matter or mixture thereof, including its formulation, which provides some therapeutic, often beneficial, effect. This includes any physiologically or pharmacologically active substance that produces a localized or systemic effect. Examples include chemotherapy agents, analgesics, steroidal anti-inflammatories, non-steroidal anti-inflammatoireanti-depressants, statins, antibiotics, anti-bacterial agents, anti-neoplastics, anti-spasmodics, modulators of cell-extracellular matrix interactions, proteins, hormones, enzymes and enzyme inhibitors, anticoagulants and/or antithrombic agents, and vasodilating agents.

The terms “antimicrobial” or “antimicrobial agent” and the like, refer to agents that are generally recognized as being useful in the treatment of microbes such as bacteria, viruses, fungi, and parasites when a therapeutically effective amount is present.

The terms “antibiotic” or “antibacterial agent” and the like, refer to agents that are generally recognized to be useful in the treatment of bacterial infections when a therapeutically effective amount is present. Suitable antibiotics include, without limitation, agents such as 2,4-diaminopyrimidines, including baquiloprim, brodimoprim, iclaprim, ormetoprim, pyrimethamine, tetroxoprim, trimethoprim; aminocoumarins including novobiocin; aminocyclitols including spectinomycin; aminoglycosides including amikacin, apramycin, arbekacin, bekanamycin, butirosin, dibekacin, dihydrostreptomycin, etimicin, fortimicins, astromicin, framycetin, gentamicin, hygromycin B, isepamicin, kanamycin, micronomicin, neomycin, netilmicin, paromomycin, plazomicin, ribostamycin, sisomicin, streptomycin, tobramycin; aminomethylcyclines including omadacycline; amphenicols including azidamfenicol, chloramphenicol, florfenicol, thiamphenicol; ansamycins including rifabutin, rifampicin, rifapentine, rifaximin; antitubercular agents including cycloserine, delamanid, ethambutol, ethionamide, morinamide, protionamide, pyrazinamide, terizidone, thioacetazone; bacteriocins, including nisin; lactam carbapenems including loracarbef biapenem, doripenem, ertapenem, faropenem, imipenem, meropenem, panipenem, razupenem, sulopenem, tebipenem, tomopenem; lactam cephalosporins including cefacetrile, cefaclor, cefadroxil, cefalexin, cefalonium, cefaloridine, cefamandole, cefapirin, cefatrizine, cefazaflur, cefazedone, cefazolin, cefcapene, cefdinir, cefditoren, cefepime, cefetamet, cefixime, cefmenoxime, cefodizime, cefonicid, cefoperazone, ceforanide, cefoselis, cefotaxime, cefotiam, cefovecin, cefozopran, cefpimizole, cefpiramide, cefpirome, cefpodoxime, cefprozil, cefquinome, cefradine, cefroxadine, cefsulodin, ceftaroline, ceftazidime, cefteram, ceftezole, ceftibuten, ceftiofur, ceftizoxime, ceftobiprole, ceftolozane, ceftriaxone, cefuroxime, cefuzonam; lactam cephamycins including cefbuperazone, cefmetazole, cefotetan, cefoxitin; lactam monobactams including aztreonam, carumonam, tigemonam; lactam oxacephems including flomoxef, latamoxef, moxalactam; lactam penicillins including amdinocillin, amoxicillin, ampicillin, apalcillin, azidocillin, azlocillin, bacampicillin, carbenicillin, ciclacillin, clemizole penicillin, cloxacillin, cyclacillin, dicloxacillin, epicillin, fenbenicillin, floxacillin, hetacillin, mecillinam, metampicillin, methicillin sodium, mezlocillin, nafcillin, oxacillin, penamecillin, penethamate hydroiodide, penicillin G, penicillin G benzathine, penicillin G procaine, penicillin N, penicillin O, penicillin V, phenethicillin potassium, piperacillin, pivampicillin, pivmecillinam, propicillin, quinacillin, sultamicillin, talampicillin, temocillin, ticarcillin; lactam in combination with lactamase inhibitors including avibactam, clavulanic acid, relebactam, sulbactam, tazobactam, vaborbactam, zidebactam; bicyclomycins including bicozamycin; bis-benzimidazoles including ridinilazole; cyclic esters including fosfomycin; fluoroquinolones including avarofloxacin, balofloxacin, besifloxacin, cinoxacin, ciprofloxacin, clinafloxacin, danofloxacin, delafloxacin, difloxacin, enoxacin, enrofloxacin, finafloxacin, fleroxacin, flumequine, garenoxacin, gatifloxacin, gemifloxacin, grepafloxacin, levofloxacin, lomefloxacin, marbofloxacin, miloxacin, moxifloxacin, nadifloxacin, norfloxacin, ofloxacin, orbifloxacin, pazufloxacin, pefloxacin, pradofloxacin, prulifloxacin, rosoxacin, rufloxacin, sarafloxacin, sitafloxacin, sparfloxacin, temafloxacin, tosufloxacin, trovafloxacin, zabofloxacin; fusidane-type including helvolic acid, fusidic acid and cephalosporin; glycolipodepsipeptides including ramoplanin; glycopeptides including avoparcin, dalbavancin, norvancomycin, oritavancin, teicoplanin, telavancin, vancomycin; glycylcyclines including tigecycline; lincosamides including clindamycin, lincomycin, pirlimycin; lipopeptides including daptomycin, surotomycin; macrolides, including azithromycin, cethromycin, clarithromycin, dirithromycin, erythromycin, fidaxomicin, flurithromycin, gamithromycin, josamycin, kitasamycin, leucomycin, miocamycin, oleandomycin, primycin, rokitamycin, rosaramicin, roxithromycin, solithromycin, spiramycin, telithromycin, tildipirosin, tilmicosin, troleandomycin, tulathromycin, tylosin, tylvalosin; nitrofurans including furaltadone, furazidin, furazolidone, nifuroxazide, nifurtoinol, nifurzide, nitrofural, nitrofurantoin, nitrofurazone; nitroimidazoles, including dimetridazole, metronidazole, ornidazole, ronidazole, secnidazole, tinidazole; oligosaccharides including avilamycin, everninomicin; polymyxins including polymyxin E (colistin), polymyxin B; polypeptides including amphomycin, bacitracin, capreomycin, enduracidin, enramycin, gramicidin, ristocetin, tuberactinomycin, tyrocidine, tyrothricin, viomycin; pseudomonic acids including mupirocin; quinoxalines including carbadox, olaquindox; sulfonamides including acetyl sulfamethoxypyrazine, chloramine-B, chloramine-T, dichloramine T, mafenide, sulfacetamide, sulfadiazine, sulfadimidine, sulfamethazine, sulfamethizole, sulfapyridine, sulfathiazole, sulfisomidine, sulfisoxazole; sulfones including dapsone sodium, sulfanilic acid; tetracyclines including chlortetracycline, clomocycline, demeclocycline, doxycycline, lymecycline, meclocycline, methacycline, minocycline, oxytetracycline, rolitetracycline, tetracycline; triazaacenaphthylene including gepotidacin, or any combination thereof.

The terms “antivirals” and “antiviral agents” refer to agents generally recognized to be useful in the treatment of viral infections when a therapeutically effective amount is present. Antiviral agents are sometimes derivatives of naturally occurring nucleotides. Examples of antiviral agents include abacavir, acyclovir, adefovir, amantadine, ampligen, amprenavir, atazanavir, atripla, baloxavir marboxil, bictegravir, boceprevir, bulevirtide, cidofovir, cobicistat, combivir, daclatasvir, darunavir, delavirdine, didanosine, docosanol, dolutegravir, doravirine, edoxudine, efavirenz, elvitegravir, emtricitabine, enfuvirtide, entecavir, etravirine, famciclovir, fomivirsen, fosamprenavir, foscarnet, ganciclovir, ibacitabine, ibalizumab, idoxuridine, imiquimod, imunovir, indinavir, lamivudine, letermovir, lopinavir, loviride, maraviroc, methisazone, moroxydine, nelfinavir, nevirapine, nexavir, nitazoxanide, norvir, oseltamivir, penciclovir, peramivir, penciclovir, peramivir, pleconaril, podophyllotoxin, raltegravir, remdesivir, ribavirin, rilpivirine, rilpivirine, rimantadine, ritonavir, saquinavir, simeprevir, sofosbuvir, stavudine, taribavirin, telaprevir, telbivudine, tenofovir alafenamide, tenofovir disoproxil, tenofovir disoproxil fumarate tenofovir, tipranavir, trifluridine, trizivir, tromantadine, truvada, umifenovir, valaciclovir, valganciclovir, vicriviroc, vidarabine, zalcitabine, zanamivir, zidovudine, or combinations thereof.

The terms “anti-inflammatory” and “anti-inflammatory agent” are also used interchangeably herein and mean and include a “pharmacological agent” and/or “active agent formulation” which, when a therapeutically effective amount is present the drug prevents or treats bodily tissue inflammation i.e., the protective tissue response to injury or destruction of tissues, which serves to destroy, dilute, or wall off both the injurious agent and the injured tissues. Examples include alclofenac, alclometasone dipropionate, alpha amylase, amcinafal, amfenac sodium, anakinra, anirolac, balsalazide disodium, bendazac, benoxaprofen, bromelains, broperamole, budesonide, carprofen, cliprofen, clobetasol propionate, clobetasone butyrate, clopirac, cortodoxone, decanoate, deflazacort, depo-testosterone, desonide, desoximetasone, dexamethasone dipropionate, diclofenac potassium, diclofenac sodium, diflorasone diacetate, diflumidone sodium, diflunisal, difluprednate, dimethyl sulfoxide, enolicam sodium, etodolac, felbinac, fenamole, fenbufen, fenclofenac, fendosal, fenpipalone, fentiazac, flazalone, flufenamic acid, flunisolide acetate, flunixin, flunixin meglumine, fluorometholone acetate, flurbiprofen, fluticasone propionate, furaprofen, halcinonide, halobetasol propionate, ibuprofen, ibuprofen aluminum, ibuprofen piconol, indomethacin, indomethacin sodium, indoprofen, isoxepac, isoxicam, ketoprofen, lofemizole hydrochloride, loteprednol etabonate, meclofenamate sodium, meclofenamic acid, mefenamic acid, mesalamine, methenolone, methenolone acetate, nabumetone, nandrolone, naproxen, naproxen sodium, naproxol, olsalazine sodium, oxaprozin, oxyphenbutazone, oxymetholone, pirfenidone, piroxicam, piroxicam cinnamate, piroxicam olamine, pirprofen, proquazone, proxazole, proxazole citrate, salsalate, stanozolol, sudoxicam, sulindac, suprofen, talniflumate, tenidap, tenidap sodium, tenoxicam, testosterone, testosterone blends, tiopinac, tixocortol pivalate, tolmetin, tolmetin sodium, triclonide, triflumidate, zidometacin, and zomepirac sodium.

### Methods of Use

This disclosure relates to methods of treating, preventing, or reversing sepsis or sepsis associated comorbidities. In certain embodiments, this disclosure relates to methods of treating, preventing, or reversing sepsis comprising administering an effective amount of a Janus kinases (JAK) inhibitor to a patient in need thereof. In certain embodiments, the JAK inhibitor is selected from baricitinib, abrocitinib, delgocitinib, fedratinib, filgotinib, oclacitinib, pacritinib, peficitinib, ruxolitinib, tofacitinib, and upadacitinib. In certain embodiments, the JAK inhibitor is administered in combination with another drug, anti-inflammatory agent, antibacterial, or antimicrobial agent.

In certain embodiments, this disclosure relates to methods of treating, preventing, or reversing sepsis-associated organ failure comprising administering an effective amount of a Janus kinases (JAK) inhibitor to a patient in need thereof. In certain embodiments, the JAK inhibitor is selected from baricitinib, abrocitinib, delgocitinib, fedratinib, filgotinib, oclacitinib, pacritinib, peficitinib, ruxolitinib, tofacitinib, and upadacitinib. In certain embodiments, the JAK inhibitor is administered in combination with another drug, anti-inflammatory agent, antibacterial, or antimicrobial agent.

In certain embodiments, this disclosure relates to methods of treating, preventing, or reversing sepsis associated encephalopathy comprising administering an effective amount of a Janus kinases (JAK) inhibitor to a patient in need thereof. In certain embodiments, the JAK inhibitor is selected from baricitinib, abrocitinib, delgocitinib, fedratinib, filgotinib, oclacitinib, pacritinib, peficitinib, ruxolitinib, tofacitinib, and upadacitinib. In certain embodiments, the JAK inhibitor is administered in combination with another drug, anti-inflammatory agent, antibacterial, or antimicrobial agent.

In certain embodiments, this disclosure relates to methods of treating, preventing, or reversing sepsis associated acute kidney injury comprising administering an effective amount of a Janus kinases (JAK) inhibitor to a patient in need thereof. In certain embodiments, the JAK inhibitor is selected from baricitinib, abrocitinib, delgocitinib, fedratinib, filgotinib, oclacitinib, pacritinib, peficitinib, ruxolitinib, tofacitinib, and upadacitinib. In certain embodiments, the JAK inhibitor is administered in combination with another drug, anti-inflammatory agent, antibacterial, or antimicrobial agent.

In certain embodiments, this disclosure relates to methods of treating, preventing, or reversing sepsis induced cardiac dysfunction comprising administering an effective amount of a Janus kinases (JAK) inhibitor to a patient in need thereof. In certain embodiments, the JAK inhibitor is selected from baricitinib, abrocitinib, delgocitinib, fedratinib, filgotinib, oclacitinib, pacritinib, peficitinib, ruxolitinib, tofacitinib, and upadacitinib. In certain embodiments, the JAK inhibitor is administered in combination with another drug, anti-inflammatory agent, antibacterial, or antimicrobial agent.

In certain embodiments, this disclosure relates to methods of treating, preventing, or reversing sepsis induced acute respiratory distress syndrome comprising administering an effective amount of a Janus kinases (JAK) inhibitor to a patient in need thereof. In certain embodiments, the JAK inhibitor is selected from baricitinib, abrocitinib, delgocitinib, fedratinib, filgotinib, oclacitinib, pacritinib, peficitinib, ruxolitinib, tofacitinib, and upadacitinib. In certain embodiments, the JAK inhibitor is administered in combination with another drug, anti-inflammatory agent, antibacterial, or antimicrobial agent.

In certain embodiments, this disclosure relates to methods of treating, preventing, or reversing hepato-splanchnic dysfunction comprising administering an effective amount of a Janus kinases (JAK) inhibitor to a patient in need thereof. In certain embodiments, the JAK inhibitor is selected from baricitinib, abrocitinib, delgocitinib, fedratinib, filgotinib, oclacitinib, pacritinib, peficitinib, ruxolitinib, tofacitinib, and upadacitinib. In certain embodiments, the JAK inhibitor is administered in combination with another drug, anti-inflammatory agent, anti-bacterial, or anti-microbial agent.

In certain embodiments, for any of the methods disclosed herein the JAK inhibitor may be administered in combination with another drug, antimicrobial agent, antibiotic, antiviral agent, antifungal agent, or anti-inflammatory agent.

In certain embodiments, the JAK inhibitor is a small molecule. In certain embodiments, the JAK inhibitor is selected from baricitinib, abrocitinib, delgocitinib, fedratinib, filgotinib, oclacitinib, pacritinib, peficitinib, ruxolitinib, tofacitinib, and upadacitinib.

### Method Using of Jak Inhibitors for Treatment, Prevention or Reversal of Sepsis and Related Comorbidities.

Sepsis is driven by the binding of pathogen associated molecular patterns (PAMPs) and damage associated molecular patterns (DAMPs), which are recognized by myeloid cells, active signal cascades resulting in transcription, and release of proinflammatory cytokines such as TNF-α, IL-1, IL-6, IL-16, and IL-1 β(7). These PAMPs include but are not limited to bacterial endotoxin lipopolysaccharides (LPS), fungal β-glucans, viral envelope glycoproteins, virions, and virion surface glycoproteins. Inflammatory cytokines activate endothelial adhesion molecules, chemokine expression, tissue factor production, and hepatic acute phase reactants. This immune dysregulation and inflammation lead to hypoperfusion, reducing oxygen delivery and therefore organ damage. There is also increased reactive oxygen species (ROS) due to the inflammation which leads to mitochondrial dysfunction, reducing ATP levels, leading to increased cellular damage.

Embodiments of this disclosure contemplates the treatment, prevention, and/or reversal of sepsis, septic shock, severe sepsis (organ failure), and the associated comorbidities including but not limited to SAE, S-AKI, SICD, ARDS, and HSD.

In certain embodiments, administration is as a monotherapy and or in combination with other agents to prevent, treat and/or reverse sepsis and its unique immune deregulatory profile through microbial infections.

In certain embodiments, for methods of treating, preventing, and/or reversing sepsis or comorbidities, antimicrobials that can be administered in combination with a Jak inhibitor, such as baricitinib, including broad-spectrum antimicrobials from multiple drug classes such as, beta-lactams, aminoglycosides, glycopeptides, and colistin.

In certain embodiments, for methods of treating, preventing, and/or reversing bacterial associated sepsis, antibiotics that can be administered in combination with a Jak inhibitor, such as baricitinib, are piperacillin and tazobactam, carbapenem, imipenem and cilastatin, cefepime, metronidazole, vancomycin, aztreonam, metronidazole, ceftriaxone, ampicillin, dexamethasone, moxifloxacin, trimethoprim and sulfamethoxazole, ceftazidime, ciprofloxacin, clindamycin, azithromycin, doxycycline, fluoroquinolone, levofloxacin, moxifloxacin, linezolid, fluoroquinolone, cefepime, and gentamicin.

In certain embodiments, for methods of treating, preventing, and/or reversing viral associated sepsis, antiviral agents that can be administered in combination with a Jak inhibitor, such as baricitinib, are acyclovir, ganciclovir, pleconaril, or combinations thereof.

In certain embodiments, for methods of treating, preventing, and/or reversing fungal associated sepsis, antifungals that can be administered in combination with a Jak inhibitor, such as baricitinib, are fluconazole, itraconazole, voriconazole, liposomal amphotericin B, amphotericin B deoxycholate, caspofungin, or combinations thereof.

In certain embodiments, for methods of treating, preventing, and/or reversing sepsis or comorbidities, pharmaceutical agents that can be administered in combination with a Jak inhibitor, such as baricitinib, are agents that target inflammation or immunomodulation, granulocyte-macrophage colony-stimulating factor, cellular factor inhibitors, small molecule inhibitors, antibody-based, peptide based, or other immune modifying agents.

In certain embodiments, for methods of treating, preventing, and/or reversing sepsis or comorbidities, pharmaceutical agents that can be administered in combination with a Jak inhibitor, such as baricitinib, are anti-TNF-α inhibitors (etanercept, infliximab, adalimumab, certolizumab pegol, golimumab), IL-6 inhibitors (tocilizumab, siltuximab), IL-1, IL-1β, IL-1α, IL-1RA inhibitors, anakinra, rilonacept, canakinumab, gevokizumab, and Y2189102.

In certain embodiments, for methods of treating, preventing, and/or reversing sepsis or comorbidities, pharmaceutical agents that can be administered in combination with a Jak inhibitor, such as baricitinib, are corticosteroids, cortisone, hydrocortisone, methylprednisolone, betamethasone, dexamethasone, or combinations thereof.

In certain embodiments, for methods of treating, preventing, and/or reversing sepsis or comorbidities, pharmaceutical agents that can be administered in combination with a Jak inhibitor, such as baricitinib, are immunotherapeutic immune-stimulators, granulocyte-macrophage colony-stimulating factor (GM-CSF), IL-7 or combinations thereof.

In certain embodiments, for methods of treating, preventing, and/or reversing sepsis or comorbidities, pharmaceutical agents that can be administered in combination with a Jak inhibitor, such as baricitinib, are vasopressors, norepinephrine, epinephrine, vasopressin, dopamine, phenylephrine, dobutamine, milrinone, or combinations thereof.

In certain embodiments, for methods of treating, preventing, and/or reversing sepsis or comorbidities, fluid resuscitation agents that can be used in combination with a Jak inhibitor, such as baricitinib, are intravenous fluids, balanced crystalloids, and saline.

### Examples

Indications of sepsis immune dysregulation include an increase in activation of both peripheral blood mononuclear cells (lymphocytes, monocytes, megakaryocytes, neutrophils, and macrophages), and central nervous cells including microglia and macrophage-like cells, specific and uniquely driven by microbial infection pathways.

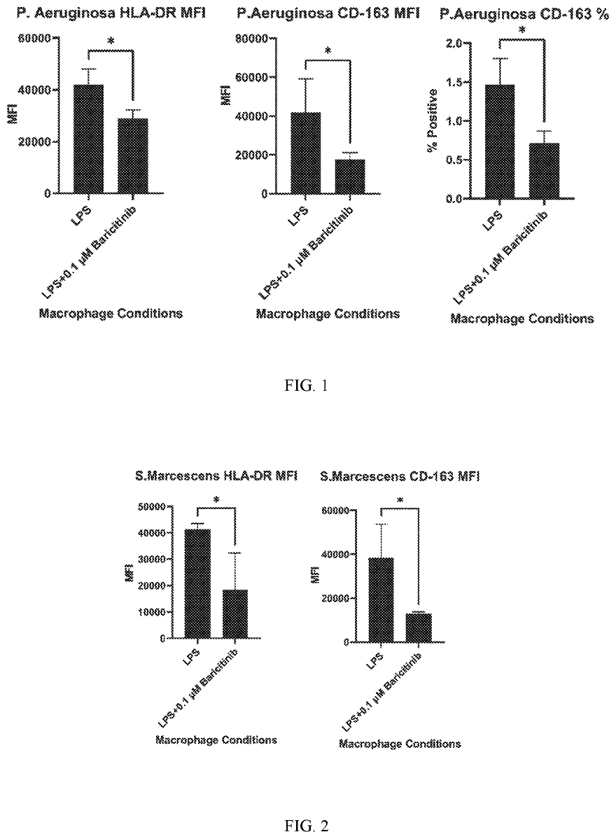

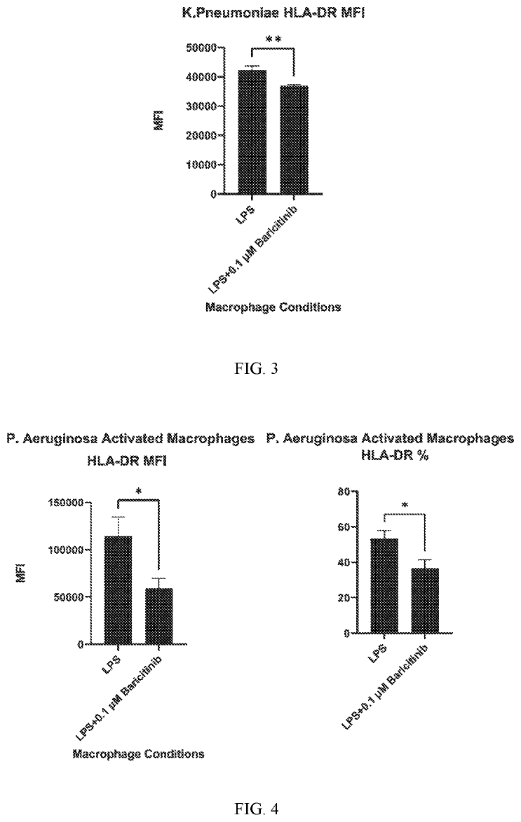

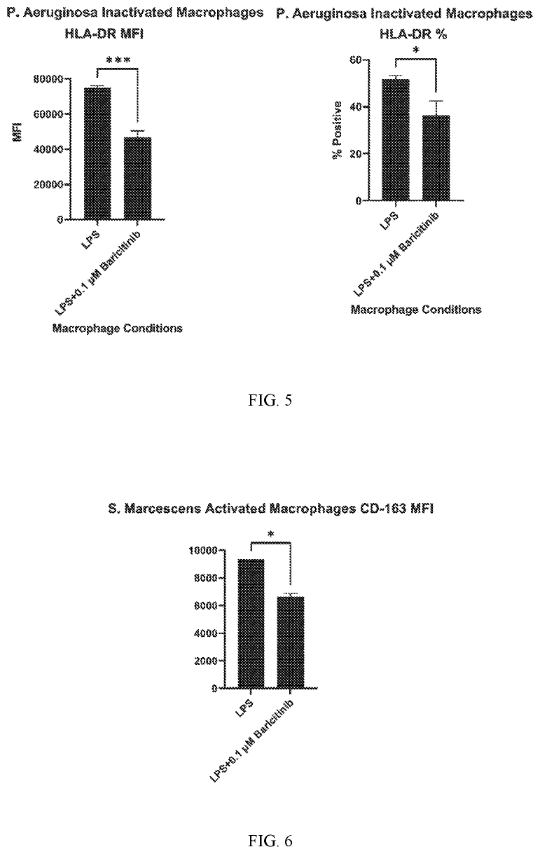

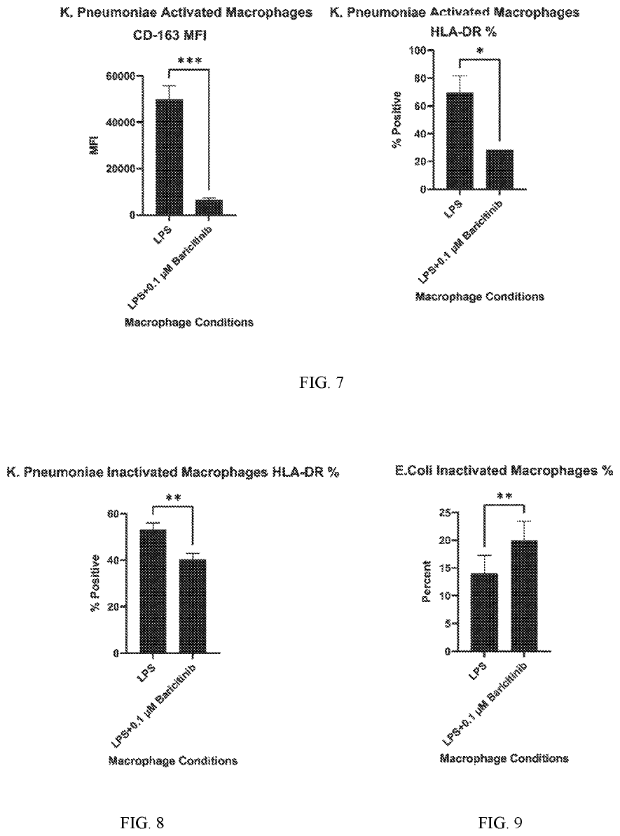

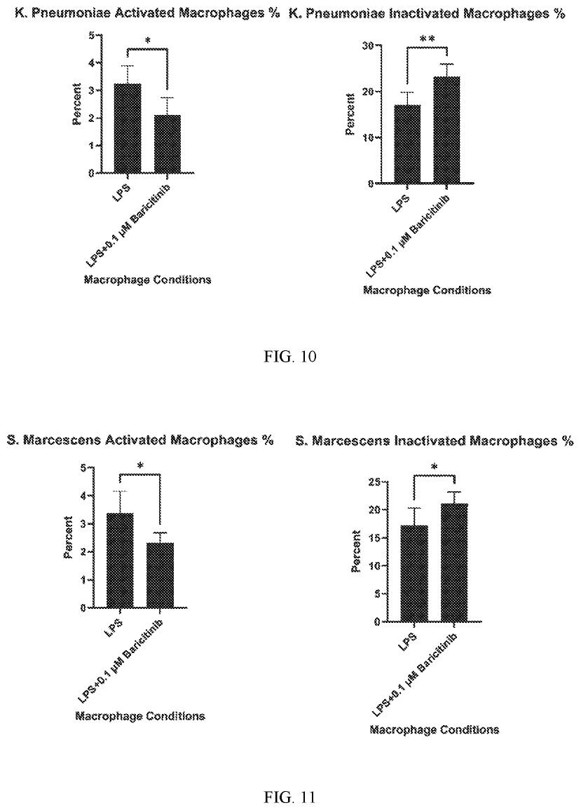

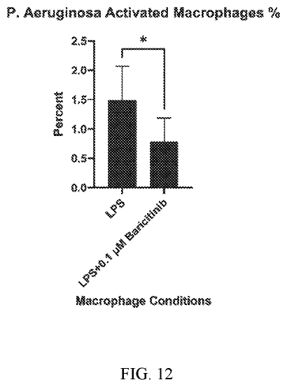

Pathogen associated molecular pathway (PAMPs) are unique to sepsis. Experiments reported herein indicate that physiologically relevant concentrations of baricitinib block immune activation and dysregulatory events in vitro that mimic the dysregulation driven by sepsis pathology. See FIGS. 1-12. These data were collected in monocyte derived macrophages (MDM). The results indicate significant reductions in cellular surface activation makers CD-163 and HLA-DR in all cell populations, as well as when gated based on activation. The results also indicate that a reduction of activated cells, and in increase of inactivated cells. These data indicate that a Jak inhibitor such as baricitinib is beneficial in sepsis for treatment or reversal of multiple indications within the septic disease phenotype.

What is claimed is:

1. A method of treating, preventing, or reversing sepsis comprising administering an effective amount of a Janus kinases (JAK) inhibitor to a patient in need thereof.

2. The method of claim 1, wherein the JAK inhibitor is selected from baricitinib, abrocitinib, delgocitinib, fedratinib, filgotinib, oclacitinib, pacritinib, peficitinib, ruxolitinib, tofacitinib, and upadacitinib.

3. The method of claim 1, wherein the JAK inhibitor is administered in combination with another drug, anti-inflammatory agent, antibacterial, or antimicrobial agent.

4. A method of treating, preventing, or reversing sepsis-associated organ failure comprising administering an effective amount of a Janus kinases (JAK) inhibitor to a patient in need thereof.

5. The method of claim 4, wherein the JAK inhibitor is selected from baricitinib, abrocitinib, delgocitinib, fedratinib, filgotinib, oclacitinib, pacritinib, peficitinib, ruxolitinib, tofacitinib, and upadacitinib.

6. The method of claim 4, wherein the JAK inhibitor is administered in combination with another drug, anti-inflammatory agent, antibacterial, or antimicrobial agent.

7. A method of treating, preventing, or reversing sepsis associated encephalopathy comprising administering an effective amount of a Janus kinases (JAK) inhibitor to a patient in need thereof.

8. The method of claim 7, wherein the JAK inhibitor is baricitinib.

9. The method of claim 7, wherein the JAK inhibitor is administered in combination with another drug, anti-inflammatory agent, antibacterial, or antimicrobial agent.

10-21. (canceled)

22. The method of claim 7, wherein the JAK inhibitor is abrocitinib.

23. The method of claim 7, wherein the JAK inhibitor is delgocitinib.

24. The method of claim 7, wherein the JAK inhibitor is fedratinib.

25. The method of claim 7, wherein the JAK inhibitor is filgotinib.

26. The method of claim 7, wherein the JAK inhibitor is oclacitinib.

27. The method of claim 7, wherein the JAK inhibitor is pacritinib.

28. The method of claim 7, wherein the JAK inhibitor is peficitinib.

29. The method of claim 7, wherein the JAK inhibitor is ruxolitinib.

30. The method of claim 7, wherein the JAK inhibitor is tofacitinib.

31. The method of claim 7, wherein the JAK inhibitor is upadacitinib.
